(19) United States
(12) Patent Application Publication
(10) Pub. No.: US 2020/0046215 A1
(43) Pub. Date: Feb. 13, 2020

(54) A MEDICAL MONITORING SYSTEM AND METHOD

(71) Applicant: GYNISUS LTD., Bat Yam (IL)

(72) Inventor: Hila FRIEDMANN, Bat Yam (IL)

(73) Assignee: GYNISUS LTD., Bat Yam (IL)

(21) Appl. No.: 16/484,483

(22) PCT Filed: Feb. 8, 2018
(86) PCT No.: PCT/IL2018/050148

§ 371 Date: Aug. 8, 2019

Related U.S. Application Data
(60) Provisional application No. 62/456,929, filed on Feb. 9, 2017.

(51) Int. Cl. A61B 1/303; A61B 8/08; A61B 8/00; A61B 5/01; A61B 8/12

(52) U.S. Cl. CPC A61B 1/303; A61B 8/485; A61B 8/4218; A61B 8/06; A61B 5/015; A61B 8/12; A61B 8/488; A61B 8/4416

(57) ABSTRACT

A medical monitoring device comprises at least one inspection sensor, configured to detect one or more events, changes or characteristic of tissue, when interfaced with a patient's body, and at least one auxiliary sensor configured to provide data regarding actuation of the device with relation to the anatomy of the patient's body.

### FIELD OF THE INVENTION

The present invention relates to the field of medical devices. More specifically the present invention relates to a system for medical monitoring.

### BACKGROUND OF THE INVENTION

Intravaginal inspections and monitoring are critical for detecting a wide variety of diseases. Cervical cancer, for example, develops in a woman's cervix due to abnormal growth of cells that have the ability to invade or spread to other parts of the body. Early detection of typical symptoms of cervical cancer may be easily missed.

Thanks to technological progress, many systems in a variety of fields are undergoing automation, i.e. are being enabled to be operated automatically and/or autonomously. For instance, autonomous medical robots are currently being developed to perform various medical procedures. In the field of obstetrics and gynecology there is need for autonomous systems capable of performing precise intravaginal inspection and monitoring.

Any autonomous system is required to undergo a learning and training phase in which the operations that are meant to be performed autonomously are performed non-autonomously and are recorded for the learning and training of the autonomous system. It is therefore an object of the present invention to provide a system for training an autonomous intravaginal inspection and monitoring system from non-autonomous intravaginal monitoring and inspections.

Furthermore, typical intravaginal monitors allow physicians to inspect and detect abnormalities on genital tissue, for example vaginal and cervix tissue. When using such a device, physicians are required to identify the position of the device with relation to the examined area in order to relate inspected tissue to medical practice and anatomical structure of the patient. Identifying the relative position of the device requires special expertise and skill that may be acquired over time, although even an expert physician might err and misidentify the relative location. Obviously when it comes to severe consequences such as cancer, there is no tolerance to such misidentification. Therefore there is need in the art for a device and method for definitely identifying the location of an intravaginal device relative to the patient's body. It is therefore an object of the present invention to provide a device for identifying the location of an intravaginal device relative to a patient's body.

Other objects and advantages of the invention will become apparent as the description proceeds.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described and illustrated in conjunction with systems, tools and methods which are meant to be exemplary and illustrative, not limiting in scope.

The present invention relates to an intravaginal monitoring device, comprising at least one inspection sensor, configured to provide at least one signal indicative of one or more characteristic of intravaginal tissue, when inserted into a patient's vagina; and at least one auxiliary sensor configured to provide data regarding actuation of the device with relation to the anatomy of the patient's body. In some embodiments, the intravaginal tissue comprises a cervix tissue. According to some embodiments, the one or more characteristic of intravaginal tissue includes pH; ionic composition; nitric oxide levels; oxygen levels; glucose levels; ATP levels; condition of cells; type of cells present; and/or mechanical property of the tissue.

According to an embodiment of the invention, the intravaginal monitoring device may be either integrally provided on an intravaginal ultrasound probe, or configured to be mounted on an intravaginal device, such as an intravaginal ultrasound probe.

According to another embodiment of the invention, the intravaginal monitoring device further comprises a processor configured to determine tissue characteristics based on the signal received from said at least one inspection sensor. In some embodiments the processor is configured to determine a dilation or deletion percentage of the cervix due to labor progression. In some embodiments the processor is configured to produce a thermal map of the intravaginal tissue; produce a topographic map of the intravaginal tissue; and/or determine the presence of pathogens according to the one or more characteristic of intravaginal tissue. According to some embodiments, the processor comprises a digital signal processor (DSP). According to another embodiment, the processor is configured to detect pathologies in the field of gynecology. In some embodiments, the processor is configured to output a medical recommendation.

According to yet another embodiment of the invention, the at least one inspection sensor is configured to transmit a signal to a tissue and to receive a returned signal indicative of a tissue characteristics. In some embodiments the tissue characteristics include tissue displacement, thermal changes, acoustic changes, chemical changes, electrical changes or any combination thereof. In yet another embodiment, the at least one auxiliary sensor is configured to detect a reflected signal pattern of pelvis bones and/or tissue, in order to identify the location of said device. In yet another embodiment of the invention, the at least one auxiliary sensor comprises a pressure sensor configured to determine the pressure applied on the tissue by said device, thereby facilitating monitoring of the elasticity of the tissue while taking into consideration the pressure applied on the tissue. In some embodiments the auxiliary sensors comprise one or more gyroscopes and/or one or more accelerometers, configured to track the location of the device and/or to navigate within the patient's body.

According to still another embodiment of the present invention, the sensors comprises at least one piezoelectric transducer; at least one ultrasonic transducer; at least one temperature sensor; at least one light sensor; at least one piezoelectric sensor; and/or at least one force sensitive resistor sensor.

In some embodiments the intravaginal device comprises a tissue excitation member configured to cause excitation of the tissue, the tissue excitation member comprising one or more mechanical devices; one or more ultrasound transducers; one or more piezoelectric crystals; and/or one or more light sources. According to an embodiment of the invention, the sensors are configured to detect one or more characteristic of the tissue as a result of the excitation.

According to an embodiment of the invention, the sensors include one or more cameras; one or more light sensors; and/or one or more RGB sensors. In some embodiments, the intravaginal device comprises a processor that is configured to generate a color image based on the signals obtained from the camera, the light sensor and/or RGB sensor.

According yet another embodiment of the invention, the intravaginal monitoring device further comprises a printed circuit board (PCB) on which the sensors are mounted or functionally connected to. In some embodiments at least part of the PCB is made of a flexible material.

In another aspect, the present invention relates to a system for intravaginal monitoring, comprising an intravaginal monitoring device as disclosed above; at least one processor in communication with said intravaginal monitoring device configured to preform analysis on data from said monitoring device; and at least one processor running machine learning algorithms on analyzed data. In some embodiments, the system for intravaginal monitoring further comprises a robot that is trained by the machine learning algorithms to autonomously perform intravaginal procedures.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

In the drawings:

FIG. 1 schematically illustrates an intravaginal monitoring device, according to an embodiment of the present invention; and

FIG. 2 shows a flowchart describing a process for training a robot to autonomously perform medical procedures, according to an embodiment of the present invention.

### DETAILED DESCRIPTION OF THE INVENTION

Reference will now be made to an embodiment of the present invention, examples of which are provided in the accompanying figures for purposes of illustration only. One skilled in the art will readily recognize from the following description that alternative embodiments of the structures and methods exemplified herein may be employed, mutatis mutandis, without departing from the principles of the invention.

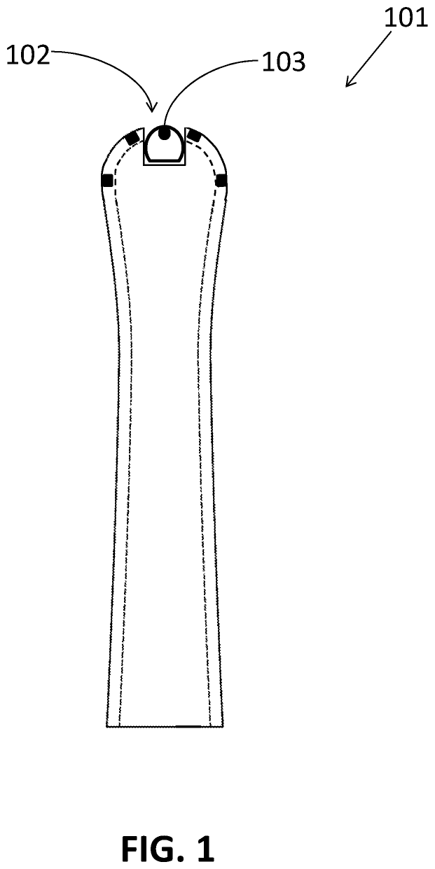

FIG. 1 schematically illustrates an intravaginal monitoring device 101, according to an embodiment of the present invention, comprising a recessed portion 102 at the distal side. A plurality of sensors (e.g. 103) are provided, some of which are housed in recessed portion 102, for detecting genital events or changes in genital tissue, and to detect data regarding actuation of the device 101 with relation to the anatomy of a patient's body. Actuation data of the device 101 may include the current location of the device relative to the anatomy of the patient's body, the force exerted by the physician while displacing the device, etc., from which an autonomous system may be trained to operate and perform various intravaginal procedures The sensors may further be configured to collect data that is allegedly irrelevant to the medical inspection or procedure being performed by the physician for the sake of training an autonomous system, such as anatomical and/or physiological data.

According to an embodiment of the present invention, device 101 is an integral part of an ultrasound intravaginal device and is integrally located at the distal end of an ultrasound probe. According to another embodiment of the present invention, device 101 a disposable apparatus configured to be attached to intravaginal devices, such as intravaginal ultrasound probes.

One or more sensors are configured to interface with the vagina, and one or more sensors are configured to interface with the cervix. Device 101 further comprises a processing unit configured to receive and process signals and data from the sensors, the processing unit comprising a signal processing module and a data processing module. Device 101 further comprises a communication module configured to transfer data to external computerized devices. According to an embodiment of the present invention, communication module comprises a wireless communication module suitable to wirelessly transfer data to external computerized devices.

According to an embodiment of the present invention, the sensors comprise one or more piezoelectric element, suitable to identify the relative location of the device 101 by detecting a reflected signal pattern of bone or tissue. The piezoelectric element may further be used to excite the cervix tissue. Initially, the piezoelectric element may be used for finding a reference point, defined as the space between the bones in the maternal pelvis.

According to another embodiment of the present invention, the sensors further comprise one or more positional sensors, e.g. accelerometer and/or gyroscope, for navigating device 101 into and in a female's vagina.

According to another embodiment of the invention, minimum clearance is maintained between the sensors depending on the geometric location of the sensors, so as to ensure minimum sensing clearance (i.e. to ensure that the entire tissue surface is measured by the relevant sensors).

According to yet another embodiment of the present invention, a flow control system is provided for controlling and supporting electrical current that is supplied to device 101. Furthermore, device 101 is configured so as to comply with known electrical and mechanical safety standards.

According to an embodiment of the present invention, the processing unit and communication module are provided on a printed circuit board (PCB) to which the sensors are connected. In some embodiments the size of the PCB is less than the geometric volume of about 4-8 mm, 26-30 mm diameter, 5.5-7.5 mm. According to an embodiment of the present invention, the PCB is placed on an ultrasound transducer. In this embodiment the PCB is flexible enough to fit to the geometric shape of the transducer, and rigid enough to dock the sensors.

According to an embodiment of the present invention, the PCB comprises one or more of the following components: one or more microprocessors serving as processing units; one or more DSP controllers serving as signal processing modules; analog and digital input ports; a wireless communication module (e.g. Wi-Fi or Bluetooth®); one or more memory elements suitable to store sensed and processed data; and one or more USB ports, e.g., for connecting cameras to device 101. According to an embodiment of the present invention, one or more of the components on the PCB are replaceable.

In the embodiments in which device 101 is placed on an ultrasound transducer, THE PCB is configured such that no interferences to the ultrasound (US) are created by the PCB and it is placed such that it does not cover the window of the transducer transmitter.

According to an embodiment of the present invention, the sensors comprise one or more pressure/force sensors, e.g. Force Sensitive Resistance (FSR). The pressure/force sensor may be used to measure pressure on the tissue applied by an ultrasound transducer and/or manually by a physician's.

According to an embodiment of the present invention, the sensors comprise one or more infrared (IR) sensors components suitable to: measure the distance between the tissue and the transducer in order to detect the tissue surface; continuously and in real time measure tissue temperature before and after excitation applied, e.g., by a Piezo transducer; build thermal maps based on the measured temperature; detect presence of viruses and bacteria; measure pH; and to continuously and in real time detect Glucose and ATP evaluation, before and after excitation, e.g., by a Piezo transducer. Resolution of the IR components is in the order of several nanometers. The power transmitted from the IR components is limited to meet regulatory requirements.

According to an embodiment of the present invention, the sensors further comprise one or more temperature sensors for calibrating the temperature measured, for example, by IR components.

According to an embodiment of the present invention, device 101 may be connected to a robotic arm and actuated thereby, as explained in detail below. The arm is designed to accurately place device 101 in a patient's body, navigate therein, and to resist device motion in the body.

According to another embodiment of the present invention, device 101 may be used in collaboration with a mobile computerized device (e.g. a tablet device) that is either dedicated to be used with device 101 or an ordinary computerized device that is adapted to collaborate therewith. Device 101 may be charged by being connected to the computerized device. In addition, a connector, which allows integration to a mechanical arm, may be attached to the computerized device and controlled thereby. According to another embodiment of the invention, the computerized device comprises circuitry for detecting and identifying a device 101, e.g. an RFID reader. In this embodiment, device 101 comprises suitable identification means (e.g. an RFID chip).

According to an embodiment of the present invention, a geolocation component (e.g. a GPS receiver) is provided so as to issue an alarm when the location of a device 101 is changed.

One or more soft and flexible protrusions comprising force sensors may be formed on the device for local measurements of applied forces. According to an embodiment of the invention, a head with force sensors is provided as a replacement plug.

According to an embodiment of the present invention, images are adjusted to the eye of the examiner/physician (hereinafter ‘user’) using a camera for detection of photoreceptors (rods and cons). The camera projects light in the visible spectrum and receives information. Using a green laser light (532 nm wavelength) to study the optical response of individual rods, several different types of laser pulses are fired at the rods and the response is measured. Before a pulse reaches the rod, the light is split into two paths. One path continues to the rod and the other goes to an avalanche photodiode (APD)—an extremely sensitive light detector capable of seeing single photons. This optical set-up may be used as a Hanbury—Brown—Twiss interferometer—which allows determining the coherence of the light arriving at the rod. Further detection of the user's sight range, the image may be adjusted using the data collected and image processing algorithm. In order to assess the suspensory ligament of lens's motion, an additional camera may be used, either located in front of the screen or a custom designed camera, which could be detached and used for other utilities.

To assess the photoreceptors amount, the examiner may place his eye as close as possible to the inner part of the camera's lens. This is performed to minimize the light intrusion to the system, in addition to proper reception of the returned light wave from the user's eye. This camera may be focused on the user's eye and therefore should bulge outwards.

According to another embodiment of the invention, the user's photoreceptors may be identified by dedicated eyeglasses comprising a camera illuminating a focused beam of laser on the user's eye. In addition, to reduce the image processing noise and to adjust to the user's eye, wavelength identification and environment lighting sensor components are provided on the eyeglasses. Eye detection may also be implemented to detect eye motion and to adjust the image properly.

According to yet another embodiment of the invention, images of the intravaginal procedure, as captured by the sensors are projected on the eyeglass's lenses. A robot may be provided on the device and allows easy, self-navigation under US or HD camera. The robot possesses image detection and allows distinguishing of tissue texture. The robot is also capable of identifying specific areas in which a genetic material (e.g. a fertilized egg) that is located at the tip of the robot may be injected for successful fertilization.

Various methods may be used for creating tissue excitation, e.g. for aiding pathological tissues recognition. According to an embodiment of the present invention, tissue excitation may be performed by:

1. using ultrasound/piezoelectric crystals/infrared (IR)/laser (located inside the upper part of device 101) or mechanically and is measured by IR or laser. In addition, displacement may be assessed on the blood cell level as well as an organ thermal map;
2. creating different levels of pulse intensity or frequency or both and measuring the reflection waves using Doppler or IR sensor, and comparing a change in motion the pulse intensity; or
3. using other methods presented herein.

According to some embodiments, acoustic and thermic properties of tissue are obtained. In order to accurately detect tissue properties (acoustic and thermic) and in particular the tissue displacement in accordance to an excitation, Red Blood Cells (RBC) movement may be detected using infrared or laser detection.

According to additional embodiments, the displacement and the thermal changes of the tissue are measure before and after of the tissue excitation. According to further embodiments, due to the mutual effect of electrical changes and chemical changes, electrical changes in the cells are checked during the tissue excitation. According to still further embodiments, electromagnetic pulses that are not in the visible spectrum are checked to find the tissue properties and specific pathogens existence.

According to some embodiments, in pursuance of ultrasound ray activation, thermal propagation (temperature changes) may be checked with the use of infrared. The IR sensor may be used to measure distance change rates as result of tissue excitation, and evaluate thermal changes in each area.

According to an embodiment of the invention, suspicious heat areas may automatically be defined as range of interest (ROI) by the system. By collecting all the data, a quality image may be captured and suspicious heat signatures in the image are automatically defined by the system as a ROI. Accordingly, an alert may be issued for accounting of the math model and then in the decision system (see description below). According to some embodiments, in order to improve the image, each ROI may be automatically taken and composed from several different directions for easier perception.

According to some embodiments, a 360 degree ultrasound transducer may be used. This transducer may use only one section of piezoelectric crystals at a time and may not be activating several of those simultaneously. The most basic aperture may contain piezoelectric crystals and sound reflectors. Their use may be averaged to a point where a minimal amount of piezoelectric crystals and movements may be made, while also maintaining a high angle count.

Processing and Adjustment of the Device/System

According to an embodiment of the invention, the ultrasound transducer detects signals that are reflected from the patient's tissue. In order to precise the results, initial measurements may detect the patient's breath and body movements. These movements are subtracted from the measured signal.

According to another embodiment of the invention, images are mapped according to areas according to the device position. Areas may be represented on this mapping according to their names. When the transducer tip starts rotating it may be possible to determine for every received image its exact location. Then, according to these locations, the areas are defined anatomically based on borders that are determined by positional sensors located on top of the device.

Currently in the field of gynecology, the reference line in the most cases is the pelvis hole, between pelvis bones. In order to perform such medical examinations, physicians identify the location manually, i.e. using their hands. According to an embodiment of the present invention, for the purpose of positioning the instrument accurately, sensors are provided ar the sides of device 101, the sensors suitable to detect a signal pattern which reflects from the pelvic bone and the tissue that wraps it. Markings of the pelvis hole may be added on the image surface and may be available for movement by the physician. According to an embodiment of the invention, after performing repeated manual detection of the pelvis hole, machine learning and deep learning methods are implemented on the image processing software, with which the procedure is fully automated.

In order to aid the physician to have a more anatomic visual experience, according to an embodiment of the invention, edge detection is utilized on B-mode images. The adjusted image may be combined with elasticity images and location markings. According to some embodiments, the enrichment processes may be performed on the local controller and computerized device (e.g. tablet device), thereby allowing fast information flow, while giving the option to view results. According to another embodiment of the invention, all data is sent to a storage device. According to yet another embodiment, the tissue elasticity module results may be enhanced by applying pressure and Doppler calculation. Since the transducer tip rotates at a calculated angle, the Doppler shift may be zero, thus results may be maximally accurate.

Doppler results depend on the angle that the transducer was while the Doppler velocity measurement was taken, while this angle should not exceed 60 degrees. Accordingly, a rotatable member may be positioned without any angle (0 degrees) between the instrument and the tissue in order to make the result more accurate and cancel the angle dependency while measuring Doppler velocity as that expressed in the equations disclosed hereinbelow. New studies show a correlation between Doppler velocity and tissue stiffness. Thus, to make the correlation between them more precise, both parameters may be used. According to an embodiment, elasticity images may too be volume images allowing obtaining of the tissue topography and stiffness.

All manner of movement may be visualized in 3D. According to some embodiments, 3D hologram containing tissue levels (different depths) may be obtained.

According to an embodiment of the invention, signal processing is performed before the image processing for each gathered data. According to some embodiments, a panoramic image of each tissue may be performed by connecting all images in a specific plane or making a 3D image by connecting them all.

According to another embodiment of the invention, the ultrasound is responsible for tissue excitation and Doppler measurements in case of tumor detection. While the IR sensor creates a standard tumor image, Doppler automatically determines if there is a greater blood flow using relevant clinical indices. In addition, the tumor size, temperature and other relevant parameters may be examined as well.

In routine scans physicians mark annotations on each image in order to restore the exact location where the image was taken. Since the instrument is positioned, and there is a track over the upper part of the instrument's movement, the annotations may be added automatically. In order to recognize in real-time the exact region, the upper part of the instrument which can rotate in 360 degrees, comprises a sensor configured to detect position (e.g. gyroscope and accelerometer) and/or by using image processing with edge detection.

Generally, the image and in particular the elasticity image may be a 3D image which may be built by using the sensors. It may illustrate tissue characteristics, such as acoustic, mechanic, thermic, optic and other characteristics. The 3D image indicates the displacement due to the ultrasound excitation of the tissue. In some cases, there is a critical layer in the receiving image which contains a lesion or another pathological finding. Multiple pulses and measuring reflections may be used, producing an image of a three-dimensional hologram and allowing displaying each layer in a different depth, region or both.

It is known that in most tumors and in other pathologies, the temperature of normal tissue is lower than in pathological tissue. Also, the pathological cells are wrapped with high vascular environment, in order to spread quickly. This can be detected by applying an excitation and creating image and Doppler image of ROI. The ROI is detected by the user or automatically by detecting markers in a combination of methods, between parameters (IR, Doppler) such as mentioned hereinabove and using image processing on the standard image.

According to an embodiment of the present invention, tumors deeper that the tissue surface (e.g. up to 2 cm in depth) may be observed, by means of engines forcing a focused IR pulse transmission and receiving it back. It would aid tremendously for accurate measurements in the area near the device.

ROIs may be colored appropriately with the use of a camera, RGB and IR sensors. Since a standard camera can only capture surface images and not tissue depth images, IR sensors are used to measure the temperature of in-depth tissue. With the temperature and a color which is obtained from the surface image, along with RGB sensors the color of the in-depth tissue may be assessed. The boundaries may be measured with the use of US. In addition, a tissue image may be taken from the camera and RGB levels may be assessed. Furthermore, a tissue thermal map may be received by an IR method. According to another embodiment of the invention, colored image may obtained by determining grey shades (to which extent each pixel taken from the ROI is red), and adding the thermal map and the boundaries received from US.

To realize and monitor the rate of change of parameters during pregnancy, they may be measured and represented in graphs. To revalue the risk of the current pregnancy with previous pregnancies, the rate of change of each parameter may be compared.

In images where a tumor is found, additional information may be presented, such as: size, volume, density, and other properties of the tumor and other tissue parameters, in addition to marking the tumor.

In medicine, especially in the fields of gynecological and obstetrics there is a big significance to measurements of organs, lesions, etc. Accordingly these measurements may be automatically identified and marked on the image. Therefore, according to an embodiment of the present invention, a thermal map is received from IR or another sensor detecting different temperatures for bones and tissues, the accurate position of the device is obtained from a positional sensor, and intensity of the ultrasound beam (which is a prediction for the returned body's density) is measured.

In order to train a robot to perform intravaginal procedures using an intravaginal device, data is collected from the abovementioned sensors and processing units and is processed by known machine learning and deep learning techniques and algorithms so as to provide training metadata for the robot. The learning may be enhanced from other physiological data regarding each patient, e.g. from patients' electronic medical record.

Robot

Once a sufficient amount of data is collected, a robot may be trained to autonomously perform intravaginal procedures. With the use of a robot whose movements are programmed according to necessary routes (as mentioned above), the displacements may be performed and new data may be gathered with high accuracy. The robot with an arm may move through the predetermined routes according to the type of diagnosis, e.g. standard test, follicular monitoring, etc.

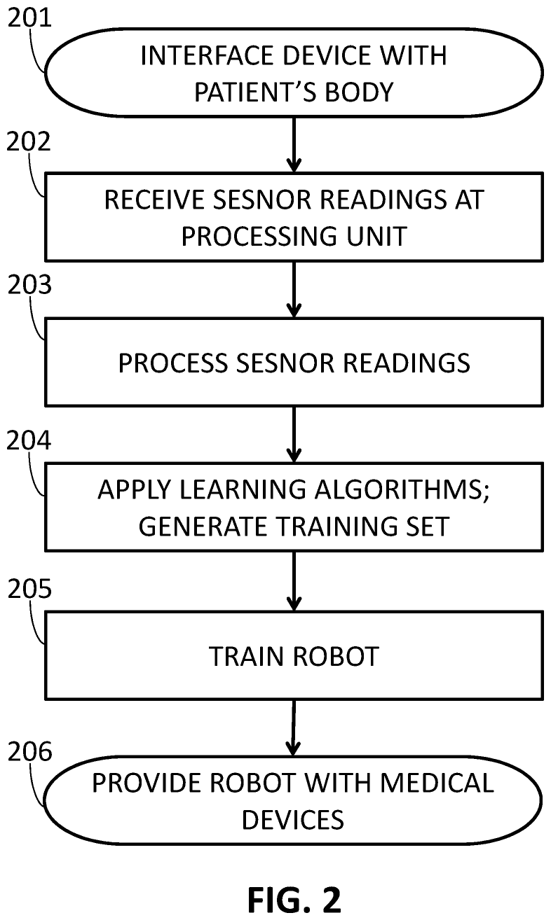

FIG. 2 shows a flowchart describing a process according to which a robot may be trained to autonomously perform medical procedures, according to an embodiment of the present invention. At the first stage 201, a medical monitoring device (e.g. 101) is interfaced with a patient's body. This may include inserting an intravaginal device (e.g. 101) or interfacing another medical device capable of detecting events, changes and characteristics of body tissue of interest. At the next stage 202 the processing unit receives readings from the sensors of the medical monitoring device, after which, at stage 203, the data is processed either by the processing unit or by a remote processor to which the sensor readings are transferred (wiredly or wirelessly). At the next stage 204 learning algorithms are applied to the readings so as to generate a training set for the autonomous robot. At the next stage 205 the robot is trained by the training set, and finally at stage 206 the robot is provided with medical devices suitable to perform various procedures that it was trained to perform.

A standard camera, a thermal camera or any other technological device may be placed on the upper side of the robot arm holding the vaginal or abdominal transducer. Planning of the route through which the robot moves may be performed by the camera as follows:
1. The robot is automatically placed on the navel either manually or with image recognition.
2. A few initial images are taken in a few locations that serve as guidelines.
3. Image processing is applied to the images gathered in order to identify the abdomen outlines (abdomen grid may be performed).
4. To grid the abdomen and for the route planning, each grid line is split into a finite number of points and an interpolation equation is calculated.
5. The received images are processed and used for a mathematical model, machine learning and decision system.

To limit the force produced by the robot, force sensors are placed on the vaginal transducer. In addition, according to patient's weight and the physician's capabilities, the robot pressing rate may be determined. This pressing value may be applied on the abdominal transducer. The map of the robot pressing rate as a function of the placement is calculated, including the tissue reaction forces.

Post processing may be performed, according to some embodiments, for example, image processing may be available (contrast, brightness, etc.) after the image was accepted. The images may be stored in the databased for further changes by the physician if such changes need be. There may be an option to stop the process and investigate a specific area much more in depth.

According to an embodiment of the invention, the gynecological system is interfaced with a home device, therefore allowing better monitoring of the women conditions and alerting about changes back to the gynecological system.

There is a need to integrate the numerical models with the system in order to demonstrate to the physician the process of thermal distribution and wave propagation. As a result, it is possible to accurately assess the tissue mechanical value (Young modulus). This may facilitate distinguishing between cysts, containing fluids and tumors. The reason for this is that fluids and pathological tissue act differently under pressure over time.

Automated Scanning

According to some embodiments, predetermined automatic options for intravaginal scanning are provided. The options include, for example:
After attaining the device's specific location, the physician may determine the ROI. This area/region may be automatically scanned in accordance to the tissues' mechanical elasticity in different depths using stepped frequencies alterations. Such method of scanning may allow observing a few tissue layers of the determined ROI.
Exemplary plains are taken according to the physicians' diagnosis for further moderation and more accurate measurements. During the image processing some organs may seem unfitting to their original size due to the scanned plain angle. This fact should be taken into account for every achieved result.
Using DSP analysis, the piezo sensor may define the bone location. If the sensor issues an appropriate alert, a transducer may be placed on that spot. Consequently, an image containing information about distances from various organs in different directions may be displayed to the physician.
After a short scan of the physician's (user's) eye pupil movement and behavior (as mentioned hereinabove), the image may be automatically adjusted. The image movement and improvement may be controlled by a self-learning system that may alter the image's resolution, contrast shading, etc. All matches may be saved for each user as his/her own preset. All changes done, if any, may be documented by the self-learning system for future use. The images' contrast, shading and consistency, in due time, may be adjusted automatically to fit the needs of the physician.
Due to the fact that men and women see and recognize different shades of grey, the self-learning system accounts to this as well. For each image, the consistency may be adjusted in accordance to the user's pre-determined database precepts and gender.

To provide precise results, ultrasound wave speeds may be changed according to the elasticity module previously assessed.

According to an embodiment of the invention, tissue stimulation may be performed by a bulge located on the tip of the transducer when the engines are turned on. The tissue movement may be measured by an IR sensor. An oscillator may be used to measure the tissue's elasticity module. The oscillator may automatically push with a force that can chang gradually. As a result, mechanical vibrations are applied to tissue. The displacement may be measured by IR and/or US (ultrasound) sensors. Comparison between the two images may be performed by motion detection. An algorithm is used to integrate the B-Mode and Doppler US in order to differentiate between cysts and specific tumors.

Application GUI

Face recognition software may be used to access the system. For each user there is a database (DB) which contains the user's patients and data. To access the DB of a fellow colleague, an account name and password is required. At first access, an explanation video and a demonstration video are provided about the system to aid the understanding of each feature.

Each elastography image comprises a quality value based on depth of the examination and the specific area in which the contact was made.

Methods of Image and Data Acquiring

Device 101 fits to the structure of the body and exploits the ability of each of the abovementioned module to evaluate the required parameters. Once device 101 is positioned, it begins to process and gather data for diagnosis. To obtain visualization, a camera is placed on the top end of the device. A user may track the camera's motion using one of the following combinations to see the ROI at any given time, and to obtain the relevant information:
A circular motion component is provided on the top side of device 101, the component controlled and actuated with a joystick hardware and software.
A per-programmed automatic motion of the camera. The camera may also gather the necessary images from each predetermined location. The per-programmed automatic motion of the camera results in the ROI being scanned in several paths. For example, scanning can be performed in a matrix manner. I.e., the camera may scan from the bottom left corner, move to the right until it reaches the bottom right corner and move slightly up to repeat the previous process, until the movement and scanning is finished at the top right corner. This method allows to obtain continuous monitoring of the ROI.
A rotating camera or a transvaginal US may be used during the procedure to monitor every motion of the device 101.
A circular motion component may be installed on the device 101 to observe ROI of half a sphere (θ=180°; ϕ=360°).

All of the obtained data is displayed on a display monitor (e.g. of the computerized device).

Diagnosis of Cervical Tissue

Several methods exist to estimate elasticity values of cervical tissue, all of which are utilized by the abovementioned components to obtain accurate results. Evaluation of tissue elasticity using these methods produces less total error in the estimations.

Method 1:

With the use of a US sensor, the distance from the cervix to the sensor is measured and the sensor is calibrated. The calculation is performed according the velocity of the signal returned from the cervical tissue with one or more options according to two states of motion—dynamic or steady state.

Steady state is achieved by a force that is applied either manually, by an oscillator, or automatically by a motor unit. The calculation is performed by using the formula Iₓ=I₀e^{-2ax}, where I₀ is the radiated intensity and Iₓ is the reflected intensity which is measured by RF signal amplitude. The signal amplitude is received from daya processing of the reflected wave. The soft tissue acoustic attenuation coefficient, μ, can be obtained from the scientific medical literature, or calculated via the formula

$$μ = ln \frac{{I}_{x}}{{I}_{0}} * \frac{1}{2 x} .$$

The soft tissue acoustic attenuation is measured by

$$α = μ [ \frac{dB}{cm * MHz} ] * x [ cm ] * f ( MHz ) .$$

Dynamic state is achieved by using the formula

$${V}_{reflected , Doppler} = \frac{{F}_{D} * c}{2 * {f}_{0} * cos θ} ,$$

where V_{reflected,Doppler} is the velocity reflected from the cervix, F_{D} is the Doppler shift (Doppler frequent), f₀ is the ultrasound frequency transmitted by the US sensor, θ is the Doppler angle and c is the speed of sound.

Furthermore, in order to find the cervical tissue elasticity coefficient E, the calculated velocity V is input into the formula

$$E = \frac{{V}_{reflected}^{2}}{ρ} ,$$

where ρ is the estimated cervical tissue density provided by scientific medical literature. For each patient the cervical density value is used according to the patient's age and week of gestation, if such values exist. Otherwise the average density value may be used.

Method 2:

With the use of a force sensor and Hooke's law

$$E = \frac{σ}{ɛ} ,$$

while stress σ and strain ε would be measured by the following methods:
1. Since stress may be calculated by the formula

$$σ = \frac{F}{A} ,$$

the external force F and contact area A must be measured. This force could be applied manually (be the physician/user) or alternatively by an oscillator or alternatively by an automatic motor unit. The force acts on cervical tissue. The external force F could be estimated by using the force sensors of device 101. A is the contact area of the device 101.
2. Strain may be obtained from image processing from the camera of device 101. The camera records with high resolution and sampling rate. The cervical tissue is compressed by device 101, after which the camera captures images and the software measures the compressed zone (ROI) during and after the tissue compression. The tissue strain resulting from the compression is calculated using the formula

$$ɛ = \frac{Δ L}{L} ,$$

where ΔL is a compressed distance, and L is an original distance. The strain rate due to the external force is measured and generated, and eventually displayed on the monitor.

Method 3:

Soft tissue elasticity value may be assessed by using thermal and RF transmitter/IR sensors/photoresistor sensors/microwave sensors and photodiodes. At first, the cervical tissue temperature T is measured by a thermal sensor and photodiode sensor. Next the measured temperature T is input to Wien's displacement law formula

$$λ = \frac{2.898 * {10}^{- 3}}{T}$$

to determine the returned optical wavelength, λ. Next, to verify the wavelength of the returned wave, an RF transmitter/IR sensor/photo resistor sensor/microwave sensor is utilized, depending on the temperature. Next LEDs located on the camera are turned on, and periodically the luminance that they produce is changed. RF transmitter/IR sensor/photoresistor sensor/microwave sensor is used in order to determine the wavelength of the reflected wave and the direction from which it came. Thus, the wavelength λ is found by using an IR sensor/phtoresistor sensor/microwave sensor.

Finally, cervical tissue properties, such as elasticity modulus, SNR, CNR, attenuation coefficient, attenuation etc., could be calculated via the following formulas:

$$α [ dB ] = μ [ \frac{dB}{cm * MHz} ] * x [ cm ] * f [ MHz ]$$

$${SNR}_{e} = \frac{μ}{α} = \frac{attenuation coefficient}{attenuation}$$

$$CNR = \frac{2 * {( {μ}_{background} - {μ}_{target} )}^{2}}{( {σ}_{background}^{2} + {σ}_{target}^{2} )}$$

where SNRₑ represents the reduction in amplitude and intensity of a signal, CNR is the Carrier to Noise Ratio, μ_{target} is the mean attenuation coefficient of a defined structure (object) in the ROI, μ_{background} is the mean attenuation coefficient of the image background suttounding the structure, σ_{background} is the general background noise expressed as a standard deviation of pixel values outside the target ROI, and σ_{target} is the mean noise expressed as a standard deviation of pixel values of a defined structure (object) in the ROI.

The elasticity may be calculated using the following formulas:

$${c}_{1} = \sqrt{\frac{E ( 1 - v )}{ρ ( 1 + v ) ( 1 - 2 v )}} \\ ⇒ E = \frac{{c}_{1}^{2} ( ρ ( 1 + v ) ( 1 - 2 v ) )}{( 1 - v )}$$

where c₁ is the longitudinal and shear (transversal) propagation velocity that could be calculated by the formula are μₜᵢₛₛᵤₑ, λ, where

$${c}_{1} = \sqrt{\frac{λ + 2 {μ}_{tissue}}{{ρ}_{tissue}}} ,$$

Lame's coefficients can be expressed as a function of the engineering constants (E—tound modulus, v—Poisson ratio).

The abovementioned methods can be used to detect an increased tissue stiffness/elasticity associated with soft tissue cancer.

According to an embodiment of the present invention, device 101 can be utilized to assess the fetal head descent during labor. In this case the same measurements and calculation that were mentioned in method 1 may be used via Doppler. In obstetrics, the gold standard of the getal head descent is the pelvis as a reference point. The point may be marked various methods:
Using a piezoelectric crystal—ultrasonic sensor to determine attenuation coefficient. Since the pelvis has a higher attenuation coefficient than the tested cervical tissue, the bone will be identified as the reference point by the dedicated software.
Using a magnet sticker stacked on the height level of the reference point. When the software identifies the magnet at certain power, the reference point is marked.
Using RFID technology.

Image and Data Processing

Image and data processing are necessary to enable analysis and storage of images after eing acquired. Multi-parametric image processing is performed by combining several different modalities. Device 101 presents computer-aided software based on US and electronic applications.

In each case, the results are received from all modalities, sent as data to the software, registered and compared by the decision system and expert system to adjust the diagnosis for high accuracy. Therefore, the software is at all times adjusted for responsiveness. The expert system can make inferences and arrive at a specific conclusion. The software can give advice and explain the logic behind the advice. The expert system provides powerful and flexible means for obtaining solutions from a variety of parameters that often cannot be dealt with together by traditional clinical methods. Accordingly, such combination of a few modalities with the expert system has high sensitivity and predictive value and can be used as a decision support system for a physician.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. Citation or identification of any reference in this application shall not be construed as an admission that such reference is available as prior art to the present invention.

1-36. (canceled)

37. A monitoring device attachable to an intervaginal device, comprising:
one or more inspection sensors configured to detected data related to an interface of the device with a tissue of an intervaginal anatomy of a patient;
one or more auxiliary sensors configured to provide data regarding a location of the device with relation to the intervaginal anatomy of the patient; and
a communication module configured to send data received from the one or more inspection sensors and the one or more auxiliary sensors to an external computerized device.

38. The monitoring device of claim 37, wherein the device is integrated with the intervaginal device.

39. The monitoring device of claim 1, wherein the device is a disposable apparatus configured to be mounted on the intervaginal device.

40. The monitoring device of claim 37, wherein the intervaginal device is an ultrasound prob.

41. The monitoring device of claim 37, further comprising a processor:
wherein the processor is configured to:
receive the data regarding the location of the device, during an intervaginal test from the one auxiliary sensors; and
receive data related to the interface of the device with the intervaginal tissue from the one or more inspection sensors;
generate an elasticity image based on the received inspection sensors data, wherein the elasticity image comprises the intervaginal tissue topography and stiffness; and
correlate between the location of the intervaginal device and the elasticity image.

42. The monitoring device of claim 41, wherein the processor is further configured to:
receive temperature measurements of the tissue, at the location, from at least one temperature sensor; and
generate a tissue thermal map form the received temperature measurements.

43. The monitoring device of claim 41, wherein the elasticity image includes a three-dimensional (3D) image of patient's tissue topography and stiffness.

44. The monitoring device of claim 41, wherein the elasticity image indicates a displacement of the tissue due to the ultrasound excitation of the tissue.

45. The monitoring device of claim 41, wherein the processor is further configured to:
receive ultrasound images taken during an ultrasound intervaginal test at the received location; and
automatically adding the received location to the ultrasound images.

46. The monitoring device of claim 45, wherein the processor is further configured to:
receive at least one of: physiological data related to the patient and electronic medical record of the patient; and
process the received ultrasound images based on at least one of: the physiological data and the electronic medical record.

47. A method of training a robot to autonomously perform an ultrasound test, to an anatomy of the patient's body, using a monitoring device, the method comprising:
receiving data regarding a location of the device, during an ultrasound test, from one auxiliary sensor, included in a monitoring device attachable to the ultrasound transducer;
receiving data related to the interface of the device with a tissue of an anatomy of the patient's body, during the ultrasound test, from one or more inspection sensors, included in the monitoring device;
generating an elasticity image based on the received inspection sensors data, wherein the elasticity image comprises the patient's tissue topography and stiffness; and
correlating between the ultrasound transducer location and the elasticity image.

48. The method of claim 47, further comprising:
receiving, from a camera associated with the monitoring device, images of the anatomy; and
analyzing the received images to identify outlines of the anatomy.

49. The method of claim 47; further comprising:
determining an upper limit for a force to be applied by the robot based on a signal received form force sensors included in the one or more inspection sensors and the patient's weight.

50. The method of claim 47, wherein elasticity image includes a three-dimensional (3D) image of patient's tissue topography and stiffness.

51. The method of claim 47, wherein the elasticity image indicates a displacement of the tissue due to the ultrasound excitation of the tissue.

52. The method of claim 47, further comprising:
receiving physiological data related to the patient; and
processing the received ultrasound images based on the physiological data.

53. A method of conducting an automated ultrasound scanning, the method comprising;
receiving an initial location in an anatomy of a patient's body;
receiving elasticity images of locations in the anatomy of patients' body, previously generated;
automatically conducting an ultrasound scanning of the anatomy of a patient's body based in the elasticity images, by operating an ultrasound transducer; and
displaying the ultrasound scanning to a user.

54. The method of claim 53, further comprising:
receiving data related to a location of a bone in the anatomy of a patient's body from one or more piezo electric sensors;
displaying an image containing information about distances of the organs from the ultrasound transducer.

55. The method of claim 53, further comprising:
receiving eye pupil movements of the user, and
automatically adjusting the display of the ultrasound scanning based on the eye pupil movements.

56. The method of claim 53, further comprising:
operating a robotic arm attached to the ultrasound transducer to place the ultrasound transducer in the anatomy.
